(19) United States
(12) Patent Application Publication
(10) Pub. No.: US 2026/0224800 A1
(43) Pub. Date: Aug. 6, 2026

(54) Medicament delivery device package assembly

(71) Applicant: SHL Medical AG, Zug (CH)

(72) Inventors: Yukiko Naoi, Norwalk, CT (US); Jacob Leon Turetsky, Norwalk, CT (US); Jeffrey Kapec, Norwalk, CT (US); Martin Michael Coyne, III, Boonton, NJ (US)

(21) Appl. No.: 19/149,894

(22) PCT Filed: Dec. 13, 2023
(86) PCT No.: PCT/EP2023/085583

§ 371 Date: Jul. 22, 2025

Related U.S. Application Data
(60) Provisional application No. 63/441,470, filed on Jan. 27, 2023.

(30) Foreign Application Priority Data
Aug. 3, 2023 (EP) 23189529.3

(51) Int. Cl. A61M 5/14; A61M 5/142; A61M 5/148

(52) U.S. Cl. CPC A61M 5/1408; A61M 5/148; A61M 2005/14268; A61M 2209/088

(57) ABSTRACT

The present disclosure provides a medicament delivery device package assembly including a package body defining an interior reservoir. The medicament delivery package further includes a plurality of medicament containers arranged inside the interior reservoir, a plurality of pressure generating modules configured to expel medicament from the plurality of medicament containers, and a base plate attached to the package body. The base plate includes a plurality of connecting slots separated from each other by a spacing, each one of the pressure generating modules being releasably attached to an associated connecting slot. The spacing is sized such that the plurality of medicament containers is arranged inside the interior reservoir to at least partly overlap each other.

### TECHNICAL FIELD

The present disclosure generally relates to medical devices for medicament administration.

### BACKGROUND

Infusion and injection are commonplace medical procedures used to deliver a wide variety of therapeutic medicines of interest for a variety of diseases. “Infusion,” “injection,” and “administration” may be used interchangeably, taking place by subcutaneous (SC), intramuscular (IM), intravenous (IV), enteral, or other routes, also terms used interchangeably. Administration route is based on a specific medication's pharmacokinetic (PK) profile, formulation components, approved regulatory labelling, individual clinical judgment, or clinical necessity.

The SC route is frequently used for administration of smaller volumes using prefilled syringes and autoinjectors. Biologic medicines are frequently administered via the SC route with these devices. SC administration is generally considered less invasive and more straightforward for patients, particularly compared to IV administration. Additionally, as physiologic uptake of medication is slower via the SC route, there is potential for improved tolerability compared to IV administration.

Given these significant advantages in safety, tolerability, and convenience, the pharmaceutical industry has invested heavily in transitioning formulations from IV to SC administration and medication administration from the clinic to the home setting. However, progress in large volume SC (LVSC) administration poses several issues that are related to the tolerability of a large volume of a single medication and the implications of this on pharmacokinetics when numerous LVSC medications are given sequentially or concurrently.

Certain biologic medications must be given in larger volumes that exceed the capacity of prefilled syringes and autoinjectors. For these medications, a larger volume device is used, such as an on-body injector (OBI, also known as a large volume infuser, “LVI,” or bolus injector) or drive unit (e.g. a motor driven pump or pressure drive) and SC needle set. Such SC needle sets include a hollow-bore needle with sharpened point for percutaneous access to patient anatomy, and a tubing set for connection to the drive unit. Each needle may be used to deliver a specific volume of medication subcutaneously, limited by patient anatomy. For some medications of moderate volume infused relatively slowly, a single needle may suffice, as in the case of OBI devices. For other medications of comparatively larger volume, anywhere from 2-8 needles may be used, the number of needles generally increasing with increasing total medication volume and/or increasing infusion rate. Multiple needles may be connected with a splitter which allows a single medication reservoir, driven by a single drive unit, to split medication flow between the medication administration needles.

When multiple medications are present, it is desirable for a clinician or pharmaceutical company to administer multiple medications to multiple injection sites, either in sequence or concurrently, while preventing inadvertent mixing or diffusion of medications between sites during administration. It is difficult to accommodate multiple medications without increasing the size of the drive unit and/or the size of the medicament delivery device; if a medicament delivery device requires a separate drive unit for each additional medication, the power requirement, size, complexity, and cost of the device in each application becomes potentially infeasible to manage, especially during final configuration and assembly of the device (i.e., dispensing) by a pharmacist. The required drive unit may consume large amounts of power, necessitating larger batteries or permanent power sources, which add weight and size of the device. Thus, there is a need for size reduction of the medicament delivery device.

### SUMMARY

In view of the above, there is a need for improved solutions to reduce the size of the medicament delivery device.

There is hence provided a medicament delivery device package assembly comprising: a package body defining an interior reservoir, a plurality of medicament containers arranged inside the interior reservoir, a plurality of pressure generating modules configured to expel medicament from the plurality of medicament containers, a base plate attached to the package body, the base plate comprising a plurality of connecting slots separated from each other by a spacing, each one of the pressure generating modules being releasably attached to an associated connecting slot, wherein the spacing is sized such that the plurality of medicament containers is arranged inside the interior reservoir to at least partly overlap each other.

The medicament delivery device package assembly can thus provide for multiple medicament containers efficiently using the interior reservoir. Hereby, the size of the medicament delivery device package assembly, or at least the package body thereof, can be reduced. That is, the space or volume of the interior reservoir can be efficiently used as the plurality of medicament containers is arranged inside the interior reservoir to at least partly overlap each other. The size of the medicament delivery device package assembly can be reduced, at least in one direction, as compared to an arrangement of the plurality of medicament containers inside the interior reservoir without such overlap. Moreover, the spacing between the connecting slots ensure proper distancing between the pressure generating modules while not excessively increasing the extension of the base plate in a direction concurrent with the spacing. In some examples, the connecting slots, and the spacing between the connecting slots, are decisive for the size of the base plate, and thereby also the size medicament delivery device package assembly, in the direction concurrent with the spacing. That is, owing to the at least partly overlapping medicament containers in the interior space, the size of the medicament delivery device package assembly may be limited to other components of the medicament delivery device package assembly than the medicament containers. The spacing between the connecting slots can be designed based on a predefined size of the base plate, or of a predefined size of the medicament delivery device package assembly. The size of the connecting slots is typically designed based on a predefined size of the pressure generating modules. As the size of pressure generating modules typically is predefined, the size of the spacing between the connecting slots are adapted in response to the predefined size of the medicament containers, such that the plurality of medicament containers is arranged inside the interior reservoir to at least partly overlap each other.

In the present disclosure, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment the package body is foldable over the plurality of medicament containers. Hereby, access to the interior reservoir e.g. for installation and removal of medicament containers is facilitated. Thus, the package body may be arrangeable in an open state in which the package body is unfolded, i.e. in which the interior reservoir is opened to the surroundings for access to the interior reservoir, and arrangeable in a closed state in which the package body is folded over the plurality of medicament containers, i.e. in which the interior reservoir is closed to the surroundings, in which access of the interior reservoir is prohibited.

According to one embodiment, the package body comprises a base portion and a cover, the base portion being attached to the base plate and the cover being foldable over the base portion to form said interior reservoir. Hereby, an efficient structure of achieving the above mentioned unfolded and folded states of the package body is provided. Thus, in the unfolded state, the cover is unfolded, and does not cover the base portion, and in the folded state, the cover is folded over the base portion, and the plurality of medicament containers arranged in the interior reservoir.

According to one embodiment, the cover is a first cover extending from the base portion on a first side thereof, and the package body further comprises a second cover extending from the base portion on a second side thereof, the second side being opposite to the first side, wherein the first and second covers are foldable over each other and the plurality of medicament containers. Hereby, the foldable structure of the package body can be improved. For example, the provision of the foldable first and second covers may achieve an increased rigidity and improved impact resistance of the package body. In some examples, the first and second covers are attachable, or lockable, to each other by magnets, a hook and loop configuration, or a snap-fit connection.

According to one embodiment, the package body comprises a plurality of pouches, each one of the pouches being configured to releasably hold an associated medicament container, wherein the plurality of pouches is arranged to at least partly overlap each other. Thus, an efficient structure for arranging the plurality of medicament containers inside the interior reservoir to at least partly overlap each other is provided. The plurality of pouches is preferably arranged in the previously mentioned base portion of the package body. A specific, or single, medicament container is typically arranged in an associated pouch. For example, the plurality of pouches is two, three or four pouches.

According to one embodiment, the package body has an extension along a centre axis from a proximal portion to a distal portion, the base plate being attached to the package body at its proximal portion, and wherein the plurality of medicament containers is arranged to at least partly overlap each other in a direction transverse the centre axis. Hereby, the extension of the plurality of medicament containers in the direction transverse the centre axis can be reduced or kept down.

According to one embodiment, the package body is defined by having a length in the longitudinal direction along the centre axis, a height in the transverse direction to the centre axis, and a thickness in the direction being transverse both the direction of the length and the height. Stated differently, the package body can be defined in a three-dimensional space by three Cartesian coordinates, x, y and z, wherein the y-axis extends along the length of the package body (along the centre axis), the z-axis extends along the height of the package body and the x-axis extends along the thickness of the package body. In other words, the plurality of medicament containers is arranged to at least partly overlap each other along the height of the package body. Thus, referring to the corresponding extension of the plurality of medicament containers arranged in the interior space of the package body, the corresponding height of the plurality of medicament containers can be reduced or kept down, typically at the expense of an increased thickness of the plurality of medicament containers.

According to one embodiment, the direction transverse the centre axis (or the height) of the package body is referred to as a vertical direction. Thus, referring to the corresponding extension of the plurality of medicament containers arranged in the interior space of the package body, the plurality of medicament containers is arranged to at least partly overlap each other in the vertical direction.

According to one embodiment, owing to the arrangement of the plurality of medicament containers to at least partly overlap in the direction transverse the centre axis, the extension of the base plate in the direction transverse the centre axis is larger as compared to the extension of the plurality of medicament containers in the direction transverse the centre axis. That is, the height of the base plate may according to one embodiment be larger as compared to the height of the plurality of medicament containers. As previously described, the spacing between the connecting slots ensure proper distancing between the pressure generating modules while not excessively increasing the extension of the base plate in the direction transverse the centre axis. The connecting slots, and the spacing between the connecting slots, may thus be decisive for the size of the base plate, and the medicament delivery device package assembly, in the direction transverse the centre axis.

It should be understood that the distal portion of the package body, or of the medicament delivery device package assembly, is here defined as further away (e.g. with reference to the fluid pathway of the medicament) from a delivery site of the medicament as compared to the proximal portion.

According to one embodiment, the plurality of medicament containers is arranged to at least partly overlap each other in the direction transverse the centre axis by 10%-70%. That is, at least 10%-70% of one medicament container overlap with a neighbouring medicament container in the direction transverse the centre axis (i.e. along the height of the package body). According to one embodiment, the plurality of medicament containers is arranged to at least partly overlap each other in the direction transverse the centre axis by 10%-50%, or 20%-60%. With reference to the previously described embodiment in which the package body comprises a plurality of pouches, the plurality of pouches is arranged to at least partly overlap each other in the direction transverse the centre axis by 10%-70%, or 10%-50%, or 20%-60%. According to one embodiment, the degree of partial overlap corresponds to the size of the pouch or medicament volume contained in each pouch.

According to one embodiment, the medicament delivery device package assembly further comprises a plurality of delivery tubes for providing medicament to an injection assembly, each one of the delivery tubes being fluidly connected to an associated medicament container, wherein the base plate comprises a plurality of tube slots arranged in juxtaposition to the connecting slots, each one of the tube slots being configured to releasably hold an associated delivery tube. Hereby, the plurality of delivery tubes can be fixedly, or reliably, held in the tube slots, while providing for an easy removal therefrom, e.g. when removing a delivery tube and/or a medicament container. For example, each tube slot may be delimited by at least one flexible tongue, the flexible tongue being configured to flex outwardly upon insertion of the associated delivery tube into the tube slot, and configured to flex back once the delivery tube is arranged in the tube slot. Hereby, the flexible tongue is configured to releasably hold the delivery tube in the tube slot.

According to one embodiment, the medicament delivery device package assembly further comprises an injection assembly for medicament administration comprising a first medicament delivery member fluidly connected to a first medicament container via a first delivery tube for a first predefined medicament administration, and a second medicament delivery member fluidly connected to a second medicament container via a second delivery tube for a second predefined medicament administration. Thus, the medicament delivery device package assembly may provide multiple medicament administrations, e.g. multiple (and possibly different) medications. The first medicament delivery member and/or the second medicament delivery member is/are preferably a needle, for example be one of a right angle needle, a straight needle, or a soft cannula. In some embodiments, the first medicament delivery member and/or the second medicament delivery member may alternatively be an intravenous connector, such as a threaded luer-lock connector.

With predefined medicament administration is meant that a predefined type of drug/medicament is to be administered at a predefined rate, for a predefined amount of time. The target patient group may also be predefined.

According to one embodiment, the plurality of medicament containers comprises a flexible container body arranged to conform to the package body. Hereby, the size of the package body holding the plurality of medicament containers can be reduced, or at least be kept as small as possible considering the size of the medicament containers. The flexible container body may house the medicant in the medicament container, and may e.g. be made of plastic. According to one example, the previously described cover(s) can be folded over the base portion and hold the plurality of medicament containers inside the interior space, in a space-efficient manner. The arrangement of the plurality of medicament containers in the interior reservoir to at least partly overlap each other is also further facilitated by the flexible container bodies, as the medicament containers may conform to each other. Furthermore, owing to the flexible container, the plurality of medicament containers may more easily be arranged in the previously described pouches. Moreover, owing to the flexible container body, the size of the medicament containers may be reduced as medicament is expelled from the medicament containers, reducing the size of the package body holding the plurality of medicament containers as the medicament administration(s) continues.

According to one embodiment, at least a part of the package body is flexible. For example, the previously described cover(s) can be flexible.

According to one embodiment, each one of the pressure generating modules comprises a pump being fluidly connected to an associated medicament container and configured to provide a suction force for a medicament inside the associated medicament container. Thus, an efficient means for expelling medicament from the plurality of medicament containers is provided. Thus, a suction force is achieved by each pump, e.g. by achieving a vacuum, or an under pressure (suppression), as compared to the pressure within the associated medicament container. Hereby, medicament contained within the medicament containers is sucked out of the medicament containers. With reference to the previous embodiment comprising a plurality of delivery tubes, the pumps are configured to suck medicament from the medicament containers into the plurality of delivery tubes. Each pump may e.g. be arranged integrated into an associated delivery tube, or be arranged at an end portion of the associated delivery tube. With reference to the previous embodiment comprising an injection assembly, the medicament administration(s) may be achieved in an efficient manner by the previously mentioned first and second medicament delivery members.

According to one embodiment, each one of the pressure generating modules comprises a pressure drive configured to provide a pressure force for a medicament inside the associated medicament container. Thus, an efficient means for expelling medicament from the plurality of medicament containers is provided. Thus, a pressure force is achieved by each pressure drive, e.g. by achieving pressure difference between outside and inside of the associated medicament container. Hereby, medicament contained within the medicament containers is pressed out of the medicament containers.

According to one embodiment, each one of the medicament containers is contained in a casing forming a cavity between an outer surface of the medicament container and an inner surface of the casing. Hereby, the pressure force by the pressure drives is induced in the cavity for each medicament container contained in the casing. The casings are arranged with the plurality of medicament containers inside the interior reservoir of the package body.

With reference to the arrangement of the plurality of medicament containers arranged inside the interior reservoir to at least partly overlap each other, the plurality of casings is typically arranged inside the interior reservoir to at least partly overlap each other. That is, as the casings house the plurality of medicament containers, both the casings and the medicament containers therein will be arranged at least partly overlapping inside he interior reservoir.

According to one embodiment, the casings are rigid casings. That is, each casing may form a rigid shell for the associated medicament container. Hereby, the medicament containers may be safely held within the casings.

According to one embodiment, the casings are flexible casings. That is, each casing may form a flexible shell for the associated medicament container. With reference to the previous embodiment in which the plurality of medicament containers comprises a flexible container body, the arrangement of the medicament containers inside the flexible casings may be referred to as a bag-in-bag medicament container. Owing to the flexible container bodies and the flexible casings, the combined size of the medicament containers and the casings may be reduced as medicament is expelled from the medicament containers, reducing the size of the package body holding the plurality of medicament containers and casings as the medicament administration(s) continues.

According to one embodiment, the base portion comprises a battery configured to power the plurality of pressure generating modules. According to another embodiment, the base portion comprises a battery configured to power a plurality of pump modules. According to another embodiment, the base portion comprises a pump.

According to one embodiment, the medicament delivery device package assembly is a wearable medicament delivery device package assembly. Hereby, a patient or user of the medicament delivery device package assembly may wear the medicament delivery device package assembly. For example, the medicament delivery device package assembly comprises a shoulder strap, or a harness, attached to the package body, enabling the medicament delivery device package assembly to be slung across the body of the patient. According to one embodiment, the medicament delivery device package assembly comprises a belt strap holder configured to be held in a belt strap of the patient, wherein the belt strap holder comprises a first attachment structure and the package body comprises a second attachments structure being configured for releasable attachment to the first attachment structure, e.g. by a male-female interlocking structure. Hereby, the package body can be easily attached and re-attached to the belt strap holder.

According to one embodiment, the medicament delivery device package assembly is configured to be wearable near to the body of the patient. That is, the medicament delivery device package assembly is a wearable over or under the clothing of the patient, without being directly attached to the patient's body, e.g. without being directly attached to the patient's skin.

According to one embodiment, each one of the connecting slots comprises a first fitting structure, wherein each one of the pressure generating modules comprises a second fitting structure, and wherein the first fitting structure of a specific connecting slot is configured to fit with the second fitting structure of a specific pressure generating module. Hereby, the pressure generating modules may be matched with corresponding connecting slots. For example, a first type pressure generating module (e.g. a relatively large pressure generating module) may comprise an associated first type second fitting structure, wherein the connecting slot is adapted to hold the first type pressure generating module by a correspondingly associated first type first fitting structure. Thus, a second type pressure generating module (e.g. a relatively small pressure generating module) having a second type second fitting structure will not fit into the first type first fitting structure associated with the first type pressure generating module.

According to one embodiment, the first fitting structure and the second fitting structure comprise magnets. Thus, the pressure generating modules may be efficiently held to the connecting slots in a releasable manner. With reference to the previously described embodiment with matching pressure generating modules and connecting slots, the first type second fitting structure may comprise a south facing magnet and the first type first fitting structure of the associated connecting slot may comprise a north facing magnet configured to magnetically attract each other. In the same example, a second type second fitting structure may comprise a north facing magnet, and the second type first fitting structure of the associated connecting slot may comprise a south facing magnet configured to magnetically attract each other. However, the south facing magnet of the first type second fitting structure will magnetically repel the south magnet of the second type first fitting structure, efficiently prohibiting the first type pressure generating module to be fitted with a second type connecting slot, discouraging mis-assembly of components of the medicament delivery device package assembly.

According to one embodiment, the medicament delivery device package assembly further comprises a reservoir sensor, wherein the reservoir sensor is arranged within the base plate, and wherein the reservoir sensor is configured to detect whether a specific pressure generating module is fitted into a specific connecting slot. For example, the magnets of the first and second fitting structures as described above may act as such sensor, or be connected to such sensor to verify magnetic attraction or magnetic repulsion.

According to one embodiment, the medicament delivery device package assembly further comprises an RFID-tag. For example, the first type first fitting structure and the first type second fitting structure described above together forms a first RFID-tag, and the second type first fitting structure and the second type second fitting structure described above together forms a second RFID-tag. If the identification of the first or second RFID-tag is not achieved, a warning may be triggered. Hereby, efficiently matching of pressure generating modules with associated connecting slots is provided while enabling warning of mis-assembly of components of the medicament delivery device package assembly, incorrect medication, and/or incorrect medication order (or notification of correct assembly, correct medication and/or correct medication order).

According to one embodiment, the package body comprises a wrapping strap arranged to hold the medicament containers in position within the interior reservoir. With reference to the previously mentioned embodiment comprising a plurality of pouches, the wrapping strap may be arranged on the outside of the pouches, to thereby hold the medicament containers in position within the pouches.

According to one embodiment, the package body is a reusable package body. A reusable package body have advantages for sustainability and patient convenience. However, they may be used over a long period time with storage in between, as in the home, or may be used frequently across multiple patients, as in the clinic setting. Particularly in the clinic setting, healthcare-associated infections (HAIs) pose risks of increasing patient treatment intensity and cross-transmission. HAIs are associated with longer hospital stays, post-discharge readmission, and higher expenses. As a result, there is a desire to avoid contamination by microbes, viruses, or fungi that may affect sterility of the medications delivered with such a package bodies.

The package body may e.g. comprise, or be comprised of, a fabric, e.g. a textile fabric. In one or more embodiments of the present disclosures, one or more fabric items, such as the package body, belt strap holder, shoulder strap, or harness, or portions thereof, may be provided with one or more of a persistently antimicrobial, antifungal, or antiviral agent. Areas that may be advantageously provided with such a coating include, by way of example and not limitation: wrapping strap for holding the medicament containers, the shoulder strap or harness. Such a coating could include fibers woven into the fabric material (e.g., silver fibers), be provided through a secondary coating, spray, or dipping operation, or by selecting an outer fabric layer featuring persistently antimicrobial, antifungal, or antiviral properties. For instance, the coating could comprise NordShield (Nordic BioTech Group Ltd.), as described in US publication number US 2021/0204552 A1 to Holopainen, or a fiber could be selected containing a similar compound, as detailed in US 2019/0271111 A1, also to Holopainen.

In one or more embodiments of the present disclosure, one or more rigid aspects of the designs, such as the base plate, connecting slots, or interfacing surfaces for the medicament administration are provided with (i.e., molded in, molded with) a compound featuring persistently antimicrobial, antifungal, or antiviral properties. Alternatively, a compound featuring persistently antimicrobial, antifungal, or antiviral properties may be applied to the molded (i.e., finished) components through secondary processes (e.g., chemical vapor deposition), spraying, or dipping processes. Areas that may be advantageously provided with such a feature include, by way of example and not limitation: connecting slots and pressure generating modules.

It should be noted that the number of pressure generating modules and the number of medicament containers of the medicament delivery device package assembly need not to be the same. For example, the medicament delivery device package assembly may comprise two pressure generating modules and three or four medicament containers. Thus, at least one of the pressure generating modules may be connected to multiple medicament containers (for medicament administration in sequence, concurrently, or contingently).

There is according to a second aspect provided a subassembly of a medicament delivery device package assembly comprising: a package body defining an interior reservoir for holding a plurality of medicament containers, a base plate attached to the package body, the base plate comprising a plurality of connecting slots separated from each other by a spacing, each one of the connecting slots being configured to releasably hold an associated pressure generating module configured to expel medicament from the plurality of medicament containers, wherein the spacing is sized to allow for the plurality of medicament containers to be arranged inside the interior reservoir by at least partly overlapping each other.

Effects and features of the second aspect of the invention are largely analogous to those described above in connection with the first aspect of the invention. Embodiments mentioned in relation to the first aspect of the invention are largely compatible with the second aspect of the invention, of which some are exemplified below.

According to one embodiment, the package body comprises a plurality of pouches, each one of the pouches being configured to releasably hold an associated medicament container, wherein the plurality of pouches is arranged to at least partly overlap each other.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 shows an example of a medicament delivery device package assembly and a subassembly thereof;

FIGS. 2-3 show another example of a medicament delivery device package assembly and a subassembly thereof;

FIG. 4 depicts an example of a medicament delivery device package assembly provided on a user;

FIG. 5 shows an example of a pressure generating module of a medicament delivery device package assembly;

FIG. 6 shows another example of a pressure generating module and its interaction with a medicament container of a medicament delivery device package assembly; and

FIG. 7 shows an example of a base plate with its connecting slots together with a pressure generating module of a medicament delivery device package assembly.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

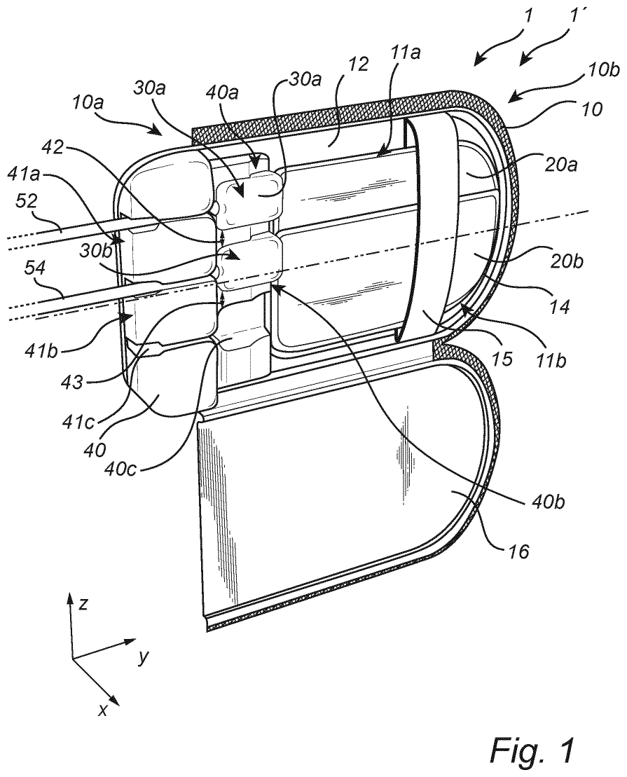

FIG. 1 shows a first example of medicament delivery device package assembly 1 according to the present disclosure.

The medicament delivery device package assembly 1 comprises a package body 10 defining an interior reservoir 12. The package body 10 is foldable over a plurality of medicament containers 20a, 20b. In the embodiment of FIG. 1, the package body comprises a base portion 14 and a cover 16. The base portion 14 is attached to a base plate 40 and the cover 16 is foldable over the base portion 14, and the medicament containers 20a, 20b, to form the interior reservoir 12 of the package body 10.

The package body has an extension along a centre axis C from a proximal portion 10a to a distal portion 10b. The package body 10 may be defined in a three-dimensional space by three Cartesian coordinates, x, y and z, wherein the y-axis extends along the length of the package body (along the centre axis C), the z-axis extends along the height of the package body 10 and the x-axis extends along the thickness of the package body 10.

The plurality of medicament containers 20a, 20b is arranged inside the interior reservoir 12 of the package body 10. In the embodiment of FIG. 1, the package body 10 comprises a plurality of pouches 11a, 11b, each one of the pouches 11a, 11b being configured to releasably hold an associated medicament container 20a, 20b. Thus, each one of the pouches 11a, 11b extends outwardly from the base portion 14 of the package body 10 (in the direction of the x-axis), thereby enabling housing of the medicament containers 20a, 20b. The pouches 11a, 11b are preferably flexible, e.g. by being made of a textile or fabric, and may completely or partly cover the medicament containers 20a, 20b. In case each pouch 11a, 11b completely covers the associated medicament container 20a, 20b, each pouch 11a, 11b is preferably provided with a flexible opening to facilitate installation and removal of the medicament container 20a, 20b.

As seen in FIG. 1, the plurality of pouches 11a, 11b is arranged to at least partly overlap each other, such that the medicament containers 20a, 20b arranged inside the pouches 11a, 11b at least partly overlap each other. As also seen in FIG. 1, the package body 10 may comprise a wrapping strap 15 arranged to hold the medicament containers 20a, 20b in position within the interior reservoir 12. In the embodiment of FIG. 1, the wrapping strap 15 is arranged on the outside of the pouches 11a, 11b, to thereby hold the medicament containers 20a, 20b in position within the pouches 11a, 11b.

The medicament delivery device package assembly 1 comprises a plurality of pressure generating modules 30a, 30b configured to expel medicament from the plurality of medicament containers 20a, 20b.

The medicament delivery device package assembly 1 comprises a base plate 40 attached to the package body 10. The base plate 40 is attached to the package body 10 at its proximal portion 10a. The base plate 40 comprises a plurality of connecting slots 40a, 40b, 40c separated from each other by a spacing 42, 43. Each one of the pressure generating modules 30a, 30b is releasably attached to an associated connecting slot 40a, 40b. In the embodiment of FIG. 1, a first connecting slot 40a releasably holds a first pressure generating module 30a, a second connecting slot 40b releasably holds a second pressure generating module 30b, and a third connecting slot 40c is presently empty, i.e. does not hold a corresponding pressure generating module. Thus, an additional pressure generating module may be added to the medicament delivery device package assembly 1, e.g. for expelling medicament from one of the already present medicament containers 20a, 20b (for redundancy or for a secondary medicament administration from the same medicament container), or to a third medicament container added to the medicament delivery device package assembly 1.

As shown in FIG. 1, the spacing 42, 43 is sized such that the plurality of medicament containers 20a, 20b, and the pouches 11a, 11b in which the medicament containers 20a, 20b are held, are arranged inside the interior reservoir 12 to at least partly overlap each other.

With reference to the Cartesian coordinate system, the plurality of medicament containers 20a, 20b is arranged to at least partly overlap each other in a direction transverse the centre axis, i.e. in the direction of the x-axis. In other words, the plurality of medicament containers 20a, 20b, and the pouches 11a, 11b, are arranged to at least partly overlap each other along the height of the package body 10.

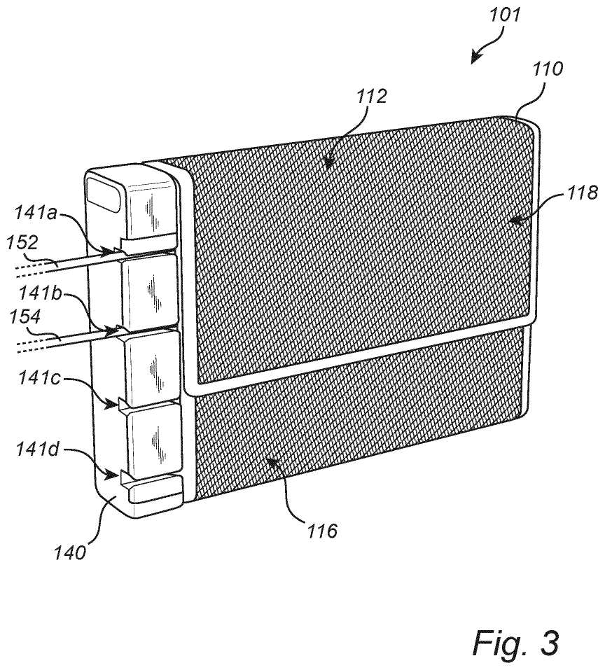

The medicament delivery device package assembly 1 comprises a plurality of delivery tubes 52, 54 for providing medicament to an injection assembly (shown in FIG. 4). Each one of the delivery tubes 52, 54 is fluidly connected to an associated medicament container 20a, 20b. Here, a first delivery tube 52 is fluidly connected to a first medicament container 20a housed in a first pouch 11a, and a second delivery tube 54 is fluidly connected to a second medicament container 20b housed in a second pouch 11b. Moreover, the base plate 40 comprises a plurality of tube slots 41a, 41b, 41c arranged in juxtaposition to the connecting slots 40a, 40b, 40c wherein each one of the tube slots 41a, 41b, 41c is configured to releasably hold an associated delivery tube 52, 54. In the embodiment of FIG. 1, a first tube slot 41a is configured to releasably hold the first delivery tube 52, and the second tube slot 41b is configured to releasably hold the second delivery tube 54. The third tube slot 41c is empty as the third connecting slot 40c does not hold a pressure generating module.

As also shown in FIG. 1, the number of pouches 11a, 11b and medicament containers 20a, 20b is smaller than the number of connecting slots 40a, 40b, 40c. Thus, and for example, one medicament container 20b may be coupled to two pressure generating modules if needed, wherein the medicament in such medicament container 20b may be used for at least two different medicament administrations (e.g. at two different locations, or injection sites, on the patient's body). However, it should be noted that the number of pouches 11a, 11b and medicament containers 20a, 20b may according to one embodiment be the same, or larger, than the number of connecting slots 40a, 40b, 40c. For example, the medicament delivery device package assembly 1 may comprise two pressure generating modules and three or four medicament containers. Thus, at least one of the pressure generating modules may be connected to multiple medicament containers.

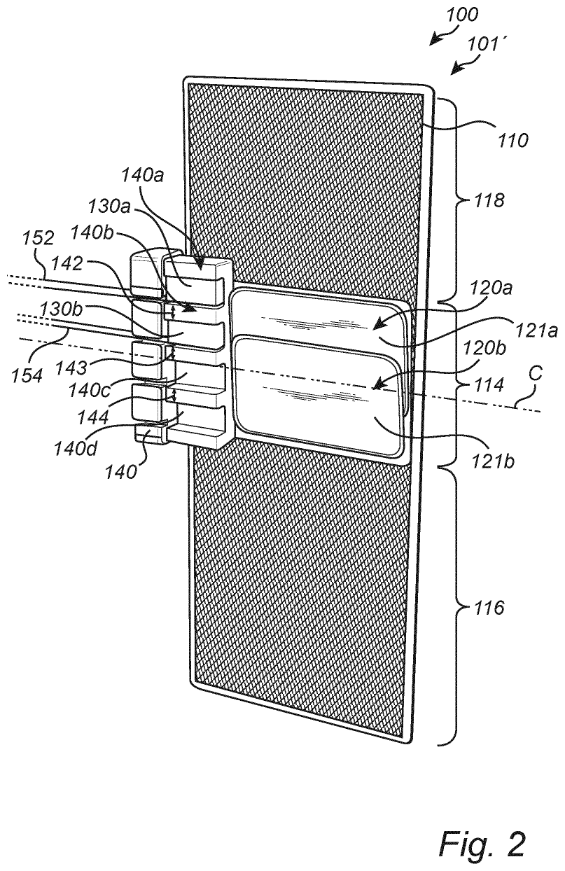

FIGS. 2 and 3 show a second example of medicament delivery device package assembly 101 according to the present disclosure.

Correspondingly to the medicament delivery device package assembly 1 of FIG. 1, the medicament delivery device package assembly 101 comprises a package body 110 defining an interior reservoir 112. The package body 112 is foldable over a plurality of medicament containers 120a, 120b. The package body 110 has an extension along a centre axis C from a proximal portion to a distal portion, and may be defined by a Cartesian coordinate system as described with reference to the medicament delivery device package assembly 1 of FIG. 1, and not repeated in detail here again.

In present embodiment, the package body 110 comprises a base portion 114 and a first cover 116 extending from the base portion 114 on a first side thereof, and a second cover 118 extending from the base portion 114 on a second side thereof, the second side being opposite to the first side. The first and second covers 116, 118 are foldable over each other and the plurality of medicament containers 120a, 120b, to form the interior reservoir 112 of the package body 110. The medicament containers 120a, 120b comprises a flexible container body 121a, 121b arranged to conform to the package body 110, and to the first and second covers 116, 118 foldable over the medicament containers 120a, 120b.

In FIG. 2, the package body 110 is arranged in an open state, or an unfolded state, in which the package body 110 is unfolded and the first and second covers 116, 118 are unfolded over the medicament containers 120a, 120b. Hence, the interior reservoir 112 is opened to the surroundings for access to the interior reservoir 112 and the medicament containers 120a, 120b. In FIG. 3, the package body 110 is arranged in a closed state, or folded state, in which the package body 110 is folded and the first and second covers 116, 118 are folded over the medicament containers 120a, 120b, and at least partly folded over each other. Hence, the interior reservoir 112 is closed to the surroundings prohibiting access to the interior reservoir 112 and the medicament containers 120a, 120b.

Correspondingly to the medicament delivery device package assembly 1 of FIG. 1, the plurality of medicament containers 120a, 120b of the embodiment in FIGS. 2-3 is arranged inside the interior reservoir 112 of the package body 110. Moreover, the medicament delivery device package assembly 101 comprises a plurality of pressure generating modules 130a, 130b configured to expel medicament from the plurality of medicament containers 120a, 120b. The medicament delivery device package assembly 101 comprises a base plate 140 attached to the package body 110 and the base portion 114 thereof. The base plate 140 comprises a plurality of connecting slots 140a, 140b, 140c, 140d separated from each other by a spacing 142, 143, 144. Each one of the pressure generating modules 130a, 130b is releasably attached to an associated connecting slot 140a, 140b.

In the embodiment of FIG. 2, a first connecting slot 140a releasably holds a first pressure generating module 130a, a second connecting slot 140b releasably holds a second pressure generating module 130b, and a third and fourth connecting slots 140c, 140d are presently empty, i.e. do not hold corresponding pressure generating modules. Thus, one or two additional pressure generating module may be added to the medicament delivery device package assembly 101, e.g. for expelling medicament from one of the already present medicament containers 120a, 120b (for redundancy or for a secondary medicament administration from the same medicament container), or to a third and/or fourth medicament container added to the medicament delivery device package assembly 101. Moreover, the medicament delivery device package assembly 101 of FIGS. 2 and 3 may comprise delivery tubes 152, 154 fluidly connected to the associated medicament containers 120a, 120b. Correspondingly to the embodiment of FIG. 1, the base plate 140 of the present embodiment may comprises corresponding plurality of tube slots for releasably holding the delivery tubes 152, 154.

In the embodiment of FIGS. 2 and 3, the package body 110 comprises no pouches to hold the medicament containers as for the embodiment of FIG. 1, but instead, the medicament containers are held in position in the interior reservoir 112 by the base plate 140 and the connection between the pressure generating modules 130a, 130b and connecting slots 140a, 140b.

As shown in FIG. 2, the spacing 142, 143, 144 is sized such that the plurality of medicament containers 120a, 120b is arranged inside the interior reservoir 112 to at least partly overlap each other. With reference to the Cartesian coordinate system, the plurality of medicament containers 120a, 120b is arranged to at least partly overlap each other in a direction transverse the centre axis, i.e. in the direction of the x-axis. In other words, the plurality of medicament containers 120a, 120b is arranged to at least partly overlap each other along the height of the package body 110.

As seen in FIG. 2, the number medicament containers 120a, 120b held in the interior reservoir 112 is smaller than the number of connecting slots 140a, 140b, 140c, 140b. Thus, and for example, at least two more medicament containers may be included in the medicament delivery device package assembly 101, providing an efficient way to adapt the medicament delivery device package assembly 101 based on the need.

Turning to FIG. 4, showing the medicament delivery device package assembly 101 of FIGS. 2 and 3 worn by a patient 100 (may also be referred to as a user 100). However, it should be noted that the patient 100 may likewise wear the medicament delivery device package assembly 1 of FIG. 1, in a corresponding embodiment.

The medicament delivery device package assembly 101 is a wearable medicament delivery device package assembly 101. In the present embodiment, the patient 100 wear the medicament delivery device package assembly 101 slung across the body of the patient. As shown in FIG. 4, the medicament delivery device package assembly comprises a shoulder strap 102 attached to the package body 110. Hereby, the medicament delivery device package assembly 101 can be wore near-body the patient 100, over or under the clothing of the patient 100, without being directly attached to the patient's 100 body, e.g. without being directly attached to the patient's 100 skin.

The medicament delivery device package assembly 101 further comprises an injection assembly 201 for medicament administration. The injection assembly 201 comprises a first medicament delivery member 203a, typically in the form a first needle 203a, fluidly connected to the first medicament container 120a (shown in FIG. 2) via the first delivery tube 152 for a first predefined medicament administration. The injection assembly 201 further comprises a second medicament delivery member 203b, typically in the form of a second needle 203b, fluidly connected to the second medicament container 120b (shown in FIG. 2) via the second delivery tube 154 for a second predefined medicament administration. Hereby, the medicament delivery device package assembly 101 may provide multiple medicament administrations, e.g. multiple (and possibly different) medications, or large volumes of the same medicament may be delivered simultaneously.

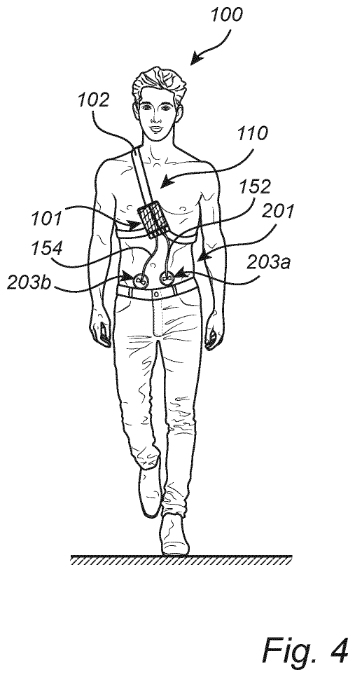

Each one of the pressure generating modules 30a, 30b 130a, 130b of the described embodiments may comprises a pump 300, shown in FIG. 5. Thus, the pump 300 is integrated in the pressure generating modules and held in the base plate 40, 140 during use. Each pump 300 is fluidly connected to an associated medicament container, such as medicament container 20a, 20b, 120a, 120b, and configured to provide a suction force for a medicament inside the associated medicament container 20a, 20b, 120a, 120b.

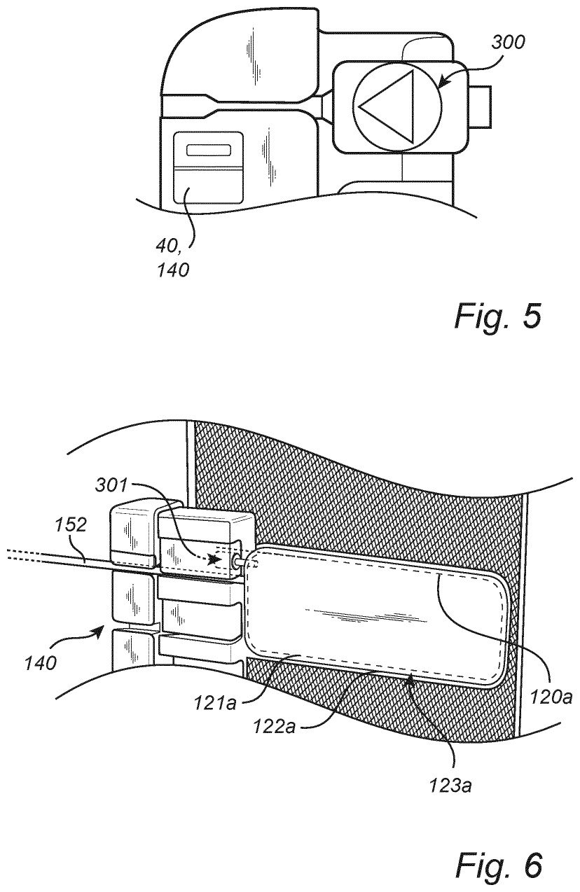

However, according to an alternative embodiment, and described in particular with reference to the medicament delivery device package assembly 101 of FIGS. 2 and 3, each one of the pressure generating modules 130a, 130b may comprises a pressure drive 301, shown in FIG. 6. Thus, the pressure drive 301 may be integrated in the pressure generating modules and held in the base plate 140 during use. As also shown in FIG. 6, the first medicament container 120a is contained in a casing 122a forming a cavity 123a between an outer surface of the medicament container 120a and an inner surface of the casing 122a. The casing 122a may be a rigid or a flexible casing. Hereby, the pressure force by the pressure drive 301 is induced in the cavity 123a for the first medicament container 120a contained in the casing 122a. Thus, the pressure drive 301 is configured to provide a pressure force for a medicament inside the first medicament container 120a. The first delivery tube 152 is in FIG. 6 arranged to be in fluid contact with the medicament inside the first medicament container 120a. As the first medicament container comprises a flexible container body 121a, the induced pressure force will press, or squeeze, the first medicament container 120a and its flexible container body 121a and thereby medicament may be pressed out of the first medicament container 120a and into the first delivery tube 154. Any other medicament container in the medicament delivery device package assembly 101 may be arranged with corresponding casing and pressure drive.

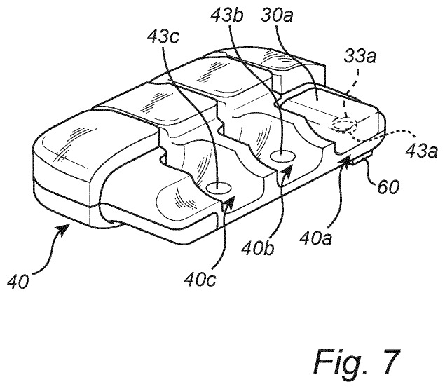

FIG. 7 shows a detailed example of the medicament delivery device package assembly 1 of FIG. 1 according to the present disclosure. In FIG. 7, the base plate 40 with its connecting slots 40a, 40b, 40c are shown together with the first pressure generating module 30a.

Each one of the connecting slots 40a, 40b, 40c comprises a first fitting structure 43a, 43b, 43c, here in the form of a magnet, i.e. a first magnet 43a, a second magnet 43b and a third magnet 43c. Correspondingly the first pressure generating module 30a comprises a second fitting structure 33a, also in the form a magnet 33a, i.e. a fourth magnet 33a. In the example of FIG. 7, only one pressure generating module 30a is shown, but any other pressure generating module comprised in the medicament delivery device package assembly, such as the second pressure generating module 30b of FIG. 1, would typically comprise a corresponding second fitting structure, or magnet. Each one of the first fitting structures 43a, 43b, 43c is associated with a specific connecting slot 40a, 40b, 40c and is configured to fit with the second fitting structure 33a of a corresponding pressure generating module 30a. For example, the first fitting structure and the second fitting structure may be configured to match a specific pressure generating module with a corresponding, and associated, connecting slot. For example, a first type first fitting structure may comprise a north facing magnet and a first type second fitting structure may comprise a south facing magnet to magnetically attract each other. As shown in FIG. 7, the first magnet 43a is e.g. a north facing magnet and the fourth magnet 33a is a south facing magnet, ensuring that the first pressure generating module 30a associated with the first type, fits the first connecting slot 43a, also associated with the first type. In the same example, a second type first fitting structure may comprise a south facing magnet, e.g. the second magnet 43b, efficiently prohibiting the first type pressure generating module 30a to be fitted into the second connecting slot 40b, associated with the second type. Hereby, discouraging mis-assembly of components of the medicament delivery device package assembly is achieved.

The base plate 40 of FIG. 7 comprises a reservoir sensor 60. The reservoir sensor 60 is arranged and configured to detect whether a specific pressure generating module, e.g. the first pressure generating module 30a being of the previously described first type, is fitted into a specific connecting slot, e.g. the first connecting slot 40a being of the previously described first type (as exemplified by the south and north facing magnets 33a, 43a). The sensor 60 may e.g. be configured to inform a patient that a pressure generating module match with an associated connecting slot, or warn the user that a pressure generating module does not match with a connecting slot. Hereby, mis-assembly of components of the medicament delivery device package assembly can be avoided.

The inventive concept may be embodied by a subassembly 1′, 101′ of a medicament delivery device package assembly 1, 101. In such subassembly 1′, 101′, the pressure generating modules 30a, 30b, 130a, 130b and the medicament containers 20a, 20b, 120a, 120b are omitted and/or removed. That is, subassembly 1′, 101′ embodies the medicament delivery device package assembly 1, 101 prior to that the pressure generating modules 30a, 30b, 130a, 130b and the medicament containers 20a, 20b, 120a, 120b have been installed.

Thus, by referring back to FIG. 1, such subassembly 1 of the medicament delivery device package assembly 1 comprises the package body 10 defining the interior reservoir 12 for holding the plurality of medicament containers 20a, 20b, and the base plate 40 attached to the package body 10 as previously described. The package body 10 comprises the plurality of pouches 11a, 11b wherein each one of the pouches 11a, 11b is configured to releasably hold an associated medicament container 20a, 20b. Thus, the base plate 40 comprises the plurality of connecting slots 40a, 40b, 40c separated from each other by the spacing 42, 43. Each one of the connecting slots 40a, 40b, 40c is configured to releasably hold an associated pressure generating module 30a, 30b configured to expel medicament from the plurality of medicament containers 20a, 20b. As described with reference to FIG. 1, the spacing 42, 43 is sized to allow for the plurality of medicament containers 20a, 20b, and the plurality of pouches 11a, 11b, to be arranged inside the interior reservoir 12 by at least partly overlapping each other.

By referring back to FIGS. 2-3, such subassembly 101 of the medicament delivery device package assembly 101 may comprises the package body 110 defining the interior reservoir 112 for holding the plurality of medicament containers 120a, 120b, and the base plate 140 attached to the package body 110 as previously described. Thus, the base plate 140 comprises the plurality of connecting slots 140a, 140b, 140c, 140d separated from each other by the spacing 142, 143, 144. Each one of the connecting slots 140a, 140b, 140c, 140d is configured to releasably hold an associated pressure generating module 130a, 130b configured to expel medicament from the plurality of medicament containers 120a, 120b. As described with reference to FIGS. 2-3, the spacing 142, 143, 144 is sized to allow for the plurality of medicament containers 120a, 120b to be arranged inside the interior reservoir 112 by at least partly overlapping each other.

The package body, and the medicament containers, may take on numerous different outer shapes, depending on the design, the pressure generating means (providing a pressure force or a suction force), number of medicament delivery members, desired injection rotation schedule between injections, the volume of medicament to be infused at a given site through a given delivery member, nature of the medicines themselves, medication regimen (e.g., such as an oncology regimen), medication administration sequence(s), need for dispersion enhancers (e.g., hyaluronidase), a desire to prevent mixing of medications at their respective injection sites, or based on results from pharmacokinetic/pharmacodynamic modelling or human clinical trial(s) of one or more medicaments separately and/or in combination.

The medicament delivery device package assemblies described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the medicament delivery device package assemblies described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the medicament delivery device package assemblies described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the medicament delivery device package assemblies described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the medicament delivery device package assemblies described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the medicament delivery device package assemblies described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the medicament delivery device package assemblies described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the medicament delivery device package assemblies described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the medicament delivery device package assemblies described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the medicament delivery device package assemblies described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the medicament delivery device package assemblies described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A medicament delivery device package assembly comprising:
a package body defining an interior reservoir;
a plurality of medicament containers arranged inside the interior reservoir;
a plurality of pressure generating modules configured to expel medicament from the plurality of medicament containers; and
a base plate attached to the package body, the base plate comprising a plurality of connecting slots separated from each other by a spacing, each one of the pressure generating modules being releasably attached to an associated connecting slot,
wherein the spacing is sized such that the plurality of medicament containers is arranged inside the interior reservoir to at least partly overlap each other.

17. The medicament delivery device package assembly of claim 16, wherein the package body is foldable over the plurality of medicament containers.

18. The medicament delivery device package assembly of claim 17, wherein the package body comprises a base portion and a cover, the base portion being attached to the base plate and the cover being foldable over the base portion to form said interior reservoir.

19. The medicament delivery device package assembly of claim 18, wherein the cover is a first cover extending from the base portion on a first side thereof, and the package body further comprises a second cover extending from the base portion on a second side thereof, the second side being opposite to the first side, wherein the first and second covers are foldable over each other and the plurality of medicament containers.

20. The medicament delivery device package assembly of claim 16, wherein the package body comprises a plurality of pouches, each one of the pouches being configured to releasably hold an associated medicament container, wherein the plurality of pouches are arranged to at least partly overlap each other.

21. The medicament delivery device package assembly of claim 16, wherein the package body has an extension along a centre axis from a proximal portion to a distal portion, the base plate being attached to the package body at its proximal portion, and wherein the plurality of medicament containers are arranged to at least partly overlap each other in a direction transverse the centre axis.

22. The medicament delivery device package assembly of claim 16, further comprising a plurality of delivery tubes for providing medicament to an injection assembly, each one of the delivery tubes being fluidly connected to an associated medicament container, wherein the base plate comprises a plurality of tube slots arranged in juxtaposition to the connecting slots, each one of the tube slots being configured to releasably hold an associated delivery tube.

23. The medicament delivery device package assembly of claim 22, further comprising an injection assembly for medicament administration comprising a first medicament delivery member fluidly connected to a first medicament container via a first delivery tube for a first predefined medicament administration, and a second medicament delivery member fluidly connected to a second medicament container via a second delivery tube for a second predefined medicament administration.

24. The medicament delivery device package assembly of claim 16, wherein the plurality of medicament containers comprises a flexible container body arranged to conform to the package body.

25. The medicament delivery device package assembly of claim 16, wherein each one of the pressure generating modules comprises a pump being fluidly connected to an associated medicament container and configured to provide a suction force for a medicament inside the associated medicament container.

26. The medicament delivery device package assembly of claim 16, wherein each one of the pressure generating modules comprises a pressure drive configured to provide a pressure force for a medicament inside the associated medicament container.

27. The medicament delivery device package assembly of claim 16, being a wearable medicament delivery device package assembly.

28. The medicament delivery device package assembly of claim 16, wherein each one of the connecting slots comprises a first fitting structure and wherein each one of the pressure generating modules comprises a second fitting structure, and wherein the first fitting structure of a specific connecting slot is configured to fit with the second fitting structure of a specific pressure generating module.

29. The medicament delivery device package assembly of claim 28, further comprising a reservoir sensor, wherein the reservoir sensor is arranged within the base plate, and wherein the reservoir sensor is configured to detect whether a specific pressure generating module is fitted into a specific connecting slot.

30. A subassembly of a medicament delivery device package assembly, comprising:
a package body defining an interior reservoir for holding a plurality of medicament containers; and
a base plate attached to the package body, the base plate comprising a plurality of connecting slots separated from each other by a spacing, each one of the connecting slots being configured to releasably hold an associated pressure generating module configured to expel medicament from the plurality of medicament containers,
wherein the spacing is sized to allow for the plurality of medicament containers to be arranged inside the interior reservoir by at least partly overlapping each other.
